SEQUENCE LISTING (8 sequences)

SEQ ID NO: 1 - DNA, 76 bp, Artificial Sequence
  Description of Artificial Sequence Synthetic oligonucleotide
gtgtgtgtgtgtgtgtgtgtgtgtgtgtgtgtgtgtgtgtcgaccgatgaatagcggtcagatccgtacctactcg

SEQ ID NO: 2 - DNA, 36 bp, Artificial Sequence
  Description of Artificial Sequence Synthetic oligonucleotide
cgagtaggtacggatctgcgtattgcgaacgactcg

SEQ ID NO: 3 - DNA, 36 bp, Artificial Sequence
  Description of Artificial Sequence Synthetic oligonucleotide
cgagtcgttcgcaatacggctgtacgtatggtctcg

SEQ ID NO: 4 - DNA, 36 bp, Artificial Sequence
  Description of Artificial Sequence Synthetic oligonucleotide
cgagaccatacgtacagcaccgctattcatcggtcg

SEQ ID NO: 5 - DNA, 80 bp, Artificial Sequence
  Description of Artificial Sequence Synthetic oligonucleotide
gtgtgtgtgtgtgtgtgtgtgtgtgtgtgtgtgtgtgtgtgtgtgtgtgtgtgtgtgtgtgtgtgtgtgtgtgtgtgtgt

SEQ ID NO: 6 - DNA, 60 bp, Artificial Sequence
  Description of Artificial Sequence Synthetic oligonucleotide
gtgtgtgtgtgtgtgtgtgtgtgtgtgtgtgtgtgtgtgtgtgtgtgtgtgtgtgtgtgt

SEQ ID NO: 7 - DNA, 20 bp, Artificial Sequence
  Description of Artificial Sequence Synthetic oligonucleotide
gtgtgtgtgtgtgtgtgtgt

SEQ ID NO: 8 - DNA, 40 bp, Artificial Sequence
  Description of Artificial Sequence Synthetic oligonucleotide
gtgtgtgtgtgtgtgtgtgtgtgtgtgtgtgtgtgtgtgt

(19) United States
(12) Patent Application Publication
(10) Pub. No.: US 2014/0335184 A1
(43) Pub. Date: Nov. 13, 2014

(54) NANOPARTICLE ASSEMBLY, PREPARATION THEREOF, AND ACTIVE MATERIAL DELIVERING COMPOSITE COMPRISING THE SAME

(71) Applicant: Samsung Electronics Co., Ltd., Suwon-si (KR)

(72) Inventors: No-kyoung PARK, Hwaseong-si (KR); Kyu-hyun IM, Yongin-si (KR); Jae-hyun HUR, Yongin-si (KR)

(73) Assignee: Samsung Electronics Co., Ltd., Suwon-si (KR)

(21) Appl. No.: 14/091,438

(22) Filed: Nov. 27, 2013

(30) Foreign Application Priority Data
Dec. 26, 2012 (KR) 10-2012-0153701

(51) Int. Cl. A61K 9/14; A61K 41/00; A61K 31/704; A61K 31/711

(52) U.S. Cl. CPC A61K 9/143; A61K 31/711; A61K 41/00; A61K 31/704
USPC 424/489; 514/44 R

(57) ABSTRACT

Provided are a nanoparticle assembly including a DNA hydrogel and gold nanoparticles, methods of preparing thereof, and an active material delivering composite including the same. The nanoparticle assembly has an excellent biocompatibility, an ability to prevent an accumulation of the nanoparticle assembly in a body, and is capable of being used to provide physical and chemical treatment.

### CROSS-REFERENCE TO RELATED APPLICATIONS

This application claims priority from Korean Patent Application No. 10-2012-0153701, filed on Dec. 26, 2012 in the Korean Intellectual Property Office, the disclosure of which is incorporated herein in its entirety by reference.

### BACKGROUND

1. Field

The present disclosure relates to nanoparticle assemblies that exhibit excellent biocompatibility and that are easily excreted out of a body, methods of preparing the same, and active material delivering composites comprising the same.

2. Description of the Related Art

Nanotechnology (NT) relates to regulating and controlling a material at the atomic or molecular level, and is suitable for creating a new material or a new device. Hence, NT is used in various fields, such as electronics, materials, communications, machinery, medicines, agriculture, energy, the environment, and the like.

A metal nanoparticle made from a metal that is commonly referred to as a precious metal, such as gold, silver, or the like, typically has excellent optical properties. This is the result of a property inherent to the metal nanoparticle, referred to as surface plasmon resonance (SPR), which is the group oscillation of the electrons of a metal nanoparticle induced by resonance with a frequency of external light (electromagnetic field). As a result of SPR, a metal nanoparticle exhibits very strong absorption, about 10,000 to about 100,000 times greater than that of a general dye molecule. As SPR absorption relaxes, a photothermal effect may occur in which the temperature increases as energy is transferred to the surrounding medium, and a new phenomenon may arise, such as surface-enhanced Raman scattering (SERS), because a very strong local electric field occurs around the metal nanoparticle due to the oscillation of electrons. When the above properties are combined and the metal nanoparticle is bonded to a particular cell, such as a cancer cell, imaging diagnosis and photothermal effect treatments may be performed simultaneously. Also, by using the fact that the metal particles induce SERS only when metal particles are close to each other, various dynamics within a cell membrane may be observed.

In particular, in order to absorb light close to near infrared rays useful for the photothermal treatment, the size of the nanoparticle needs to be greater than or equal to 100 nm. On the other hand, a nanoparticle injected into a body for treatment needs to be excreted out of the body because, for example, in the case of a gold nanoparticle, gold may clog pores in the body and ultimately destroy tissue, even though the gold itself is not toxic.

In addition, in order to maximize the SERS effect, there is a need for nanoparticles having a big particle size or which are densely arraigned so as to have a narrow space therebetween.

### SUMMARY

One or more embodiments provide nanoparticle assemblies having excellent biocompatibility and which are easily excreted out of a body.

One or more embodiment also provide methods of preparing the nanoparticle assemblies.

One or more embodiments also provide active material delivering composites capable of easily delivering active materials, such as medicines or contrast media.

According to an aspect of an embodiment, there is provided a nanoparticle assembly including a DNA hydrogel; and nanoparticles having a positively charged surface, wherein the nanoparticles are coupled to the DNA hydrogel by electrostatic attraction.

According to an aspect of another embodiment, there is provided a method of preparing a nanoparticle assembly, the method including preparing a DNA hydrogel; preparing a solution comprising nanoparticles having a positively charged surface; and coupling the nanoparticles having the positively charged surface and the DNA hydrogel by adding the solution to the DNA hydrogel.

According to an aspect of another embodiment, there is provided an active material delivering composite, the active material delivering composite including a DNA hydrogel; nanoparticles having a positively charged surface; and an active material coupled to at least one of the DNA hydrogel and the nanoparticles.

### BRIEF DESCRIPTION OF THE DRAWINGS

The and/or other aspects will become apparent and more readily appreciated from the following description of the embodiments, taken in conjunction with the accompanying drawings in which:

FIGS. 1A and 1B schematically illustrate an X-shaped branched DNA used for preparing a DNA hydrogel, according to an embodiment;

FIGS. 2A and 2B schematically illustrate a preparation process of a DNA hydrogel, according to an embodiment;

FIG. 3 schematically illustrates a nanoparticle assembly according to an embodiment;

FIG. 4 schematically illustrates an active material delivering composite according to an embodiment;

FIGS. 5A and 5B illustrate a result of a gel electrophoresis of a DNA hydrogel;

FIGS. 6A and 6B schematically illustrate a nanoparticle having a positively charged surface;

FIG. 7 illustrates the presence and absence of coupling a quantum dot and a DNA hydrogel;

FIGS. 8A, 8B and 8C illustrate the presence and absence of coupling nanoparticles and a DNA hydrogel;

FIGS. 9A and 9B are absorption spectra of nanoparticle assemblies according to Examples and Comparative Examples;

FIGS. 10A, 10B, 10C and 10D are scanning electron microscope images of a nanoparticle assembly prepared according to an embodiment;

FIGS. 11A and 11B are transmission electron microscope (TEM) images of a nanoparticle assembly prepared according to Example 2 and an aggregate of nanoparticles for comparison;

FIG. 12 is a graph representing energy dispersive spectroscopy data of a nanoparticle assembly according to an embodiment;

FIG. 13 is an absorption spectrum of a nanoparticle assembly according to an embodiment;

FIGS. 14A, 14B and 14C are dark field microscopic images of a nanoparticle assembly after a delivery into cells according to an embodiment;

FIGS. 15A and 15B schematically illustrate coupling and excretion of an active material of an active material delivering composite according to an embodiment; and

FIGS. 16A and 16B are graphs illustrating a measurement of a decrease in a fluorescence of the active material delivering composite according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

According to an aspect of the present inventive concept, a nanoparticle assembly includes a DNA hydrogel and nanoparticles having a positively charged surface, wherein the nanoparticles are coupled to the DNA hydrogel by electrostatic attraction.

The nanoparticle assembly according to an embodiment includes the nanoparticles having a positively charged surface based on the DNA hydrogel. As a result, the nanoparticle assembly has an excellent biocompatibility and is easily coupled and decoupled.

The term “DNA hydrogel” as used herein generally refers to a gel formed by coupling branched DNA and forming a three-dimensional structure.

The DNA hydrogel in the nanoparticle assembly according to an embodiment may be formed by cross-linking at least one of X-DNA, Y-DNA, and T-DNA. The DNA hydrogel may be a nanogel having a diameter of from about 20 nm to about 500 nm. Generally, the nanogel includes a chemically or physically cross-linked synthetic polymer or biopolymer, and the diameter of the nanogel ranges from about tens of nanometers to about hundreds of nanometers. Holes in the nanogel may be filled with small molecules or macromolecules, and the swelling, degradation, and chemical action may be controlled. With respect to a nanoparticle assembly according to an embodiment, when the DNA hydrogel is the size of a nanogel, the DNA hydrogel may be easily injected into a body.

The size and porosity of the DNA hydrogel may be closely controlled by controlling the initial concentration and type of branched DNA building block used to form the DNA hydrogel. The DNA hydrogel has a porous structure and has a negative charge resulting from the phosphate group in the DNA backbone.

The nanoparticles having a positively charged surface in the nanoparticle assembly according to an embodiment may exist in the form of a cluster having an average diameter of from about 20 nm to about 500 nm. The cluster form is not a mass of aggregated nanoparticles, but is instead an assembly of nanoparticles that each maintain the form of an individual particle.

The nanoparticle assembly according to an embodiment may contain a nanoparticle cluster having a desired size arrived at by controlling the relative amounts of the DNA hydrogel and the nanoparticles. Specifically, a negatively charged DNA hydrogel and positively charged nanoparticles couple together due to electrostatic attraction, and a cluster having a desired size may be formed as the positively charged nanoparticles couple to the DNA hydrogel and assemble together until a certain size is achieved.

The nanoparticles having a positively charged surface may include a metal, a semiconductor, or a quantum dot.

The metal may be at least one selected from gold, silver, copper, platinum, iron, palladium, and an alloy thereof.

The semiconductor may be at least one selected from graphene or a carbon nanotube.

The quantum dot may be at least one selected from CdS, CdSe, and CdTe.

The nanoparticles having a positively charged surface may have a photothermal effect.

The nanoparticles having a positively charged surface may include a material capable of emitting light such as fluorescent light.

The nanoparticles having a positively charged surface may charge the surfaces of the nanoparticles by including therein a positive ion ligand. The positive ion ligand may be at least one selected from an amine (primary-, secondary-, and/or tertiary-), a cationic surfactant, and a cationic macromolecule.

As the cationic surfactant, cetyltrimethylammonium bromide (CTAB), cetyl trimethylammonium chloride (CTAC), cetylpyridinium chloride (CPC), benzalkonium chloride (BAC), benzethonium chloride (BZT), 5-bromo-5-nitro-1,3-dioxane, dimethyldioctadecylammonium chloride, or dioctadecyldimethylammonium bromide (DODAB) may be used.

As the cationic macromolecule, a lipoic acid derivative may be used.

The nanoparticles having a positively charged surface may be present in an amount of from about 0.05 moles to about 10 moles for every 1 mole of the X-, Y-, or T-DNA building block used in the synthesis of the DNA hydrogel. When the amount is within the range above, the size of the cluster formed by the nanoparticles having a positively charged surface may be controlled within an optimal range.

The nanoparticle assembly according to an embodiment may be used in various fields, such as photothermal treatment, drug delivery, and the like.

For example, gold nanoparticles emit heat after absorbing light, and this property may be used in the treatment of diseases such as cancer by destroying specific cells. In such a case, in order to improve treatment efficiency, exposure to long-wavelength light having a good biotransmissivity is advantageous and, accordingly, gold nanoparticles having a high absorption with respect to the long-wavelength light are also advantageous. In the case of commonly used spherical gold nanoparticles, as the size of the gold nanoparticle increases, the wavelength absorbed by the gold nanoparticle increases. The size of the gold nanoparticle for absorbing light close to near infrared rays useful for the photothermal treatment is at least 100 nm. However, it is almost impossible to naturally excrete a gold nanoparticle that has a size of about 100 nm. Thus, because the gold particle is not excreted and continues to exist in the body, it may act as a hazard.

However, the nanoparticles having a positively charged surface in the nanoparticle assembly according to an embodiment disclosed herein may form a cluster having an average diameter of about 20 nm to about 500 nm. Despite the fact that the nanoparticles forming a cluster in the DNA hydrogel may absorb the long-wavelength light, the DNA hydrogel may decompose, thereby decomposing the cluster into individual nanoparticles that are capable of being excreted from the body, thereby solving the problem of toxicity.

In the nanoparticle assembly according to an embodiment, the nanoparticles in the DNA hydrogel may have an optical property similar to nanoparticles having a size greater than or equal to 100 nm through an electrostatic coupling of small nanoparticles having a diameter of from a few nanometers to 10 nm. As a result of having such an optical property, the nanoparticle assembly including the nanoparticles forming a clusters coupled to the DNA hydrogel may be used as a non-toxic medium for a photothermal treatment, which is a rising new technology useful for future cancer treatment.

Provided herein is a nanoparticle assembly capable of natural decomposition within the body, something that has not previously been possible using traditional technology. The optical properties of the nanoparticle assembly vary depending on the size and density of the nanoparticle assembly. A technology for controlling the size and the density of the nanoparticle assembly may be helpful in developing an efficient photothermal treatment medicine. The size and the density of the nanoparticle assembly are controlled by the size of the DNA hydrogel to which each nanoparticle is coupled, and by the ratio of the concentration of DNA building block to the concentration of the nanoparticles.

A method of preparing the nanoparticle assembly, according to another aspect, includes preparing the DNA hydrogel; preparing a solution of the nanoparticles having a positively charged surface; and coupling the nanoparticles having a positively charged surface to the DNA hydrogel by adding the solution.

According to an embodiment, the nanoparticles having a positively charged surface may couple to the DNA hydrogel via electrostatic attraction.

Preparing the DNA hydrogel includes preparing a branched DNA by hybridizing single-stranded DNAs; and cross-linking the branched DNAs. Cross-linking the branched DNAs may be performed using an enzyme such as a ligase, by using a functional group capable of covalent bonding, or the like.

When the branched DNA is cross-linked using a T4 ligase, a three-dimensional DNA hydrogel forms as the branched DNAs are cross-linked. The method of preparing the DNA hydrogel is described in detail in an article in Nature Materials (“Enzyme-catalysed assembly of DNA hydrogel,” Sep. 24, 2006, pages 797-801), the contents of which are incorporated herein by reference.

The branched DNA may be an X-shaped branched DNA, a Y-shaped branched DNA, or a T-shaped branched DNA.

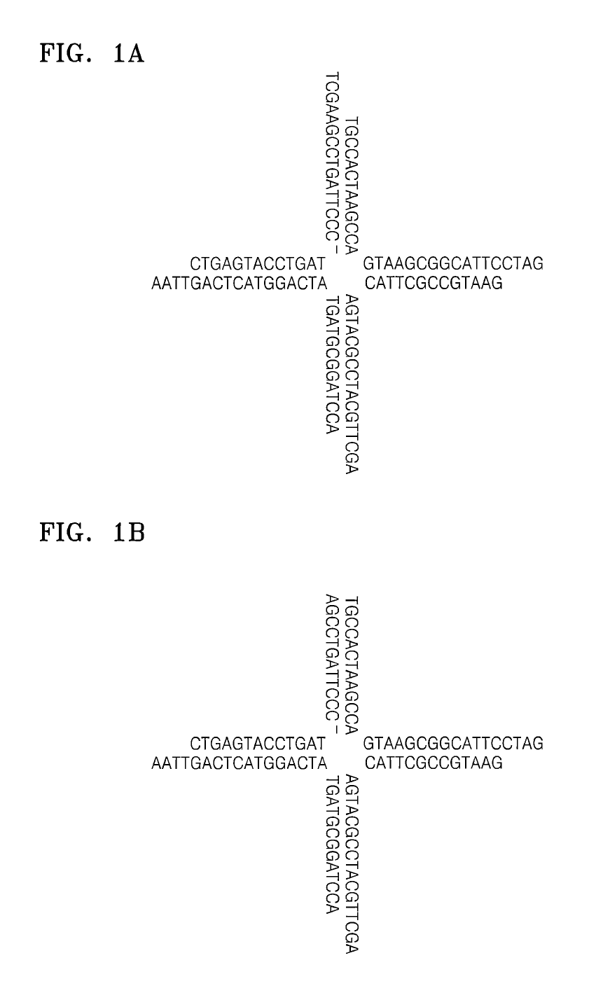

The branched DNA includes DNA having a deficiency in part of a sticky end. Due to the deficiency in part of the sticky end, the cross-linking progresses until a certain point and then stops, thereby obtaining a nanogel form DNA hydrogel having a size of from about 20 nm to about 500 nm. The DNA having the deficiency in part of its sticky end may be an X-shaped branched DNA. FIGS. 1A and 1B schematically illustrate an X-shaped branched DNA (FIG. 1A) having four sticky ends and an X-shaped DNA (FIG. 1B) having a deficiency in a part of a sticky end. In the case of an X-shaped branched DNA having a deficiency in part of the sticky end, the cross-linking may be performed until an optimal nanogel-sized DNA hydrogel, according to an embodiment, is formed.

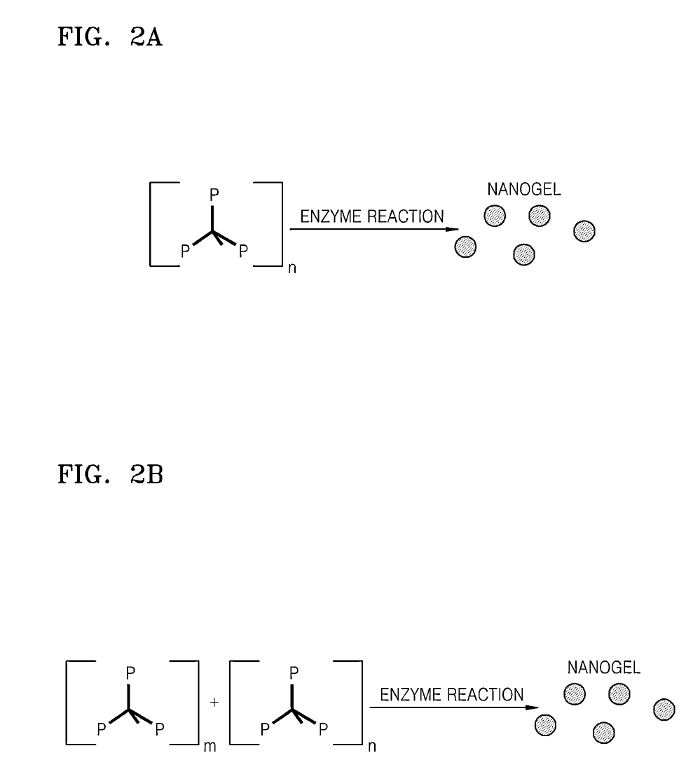

FIGS. 2A and 2B schematically illustrate a preparation process for a DNA hydrogel according to an embodiment.

Generally speaking, an X-shaped branched DNA has sticky ends including phosphate groups at four ends. However, some (for example, n in FIGS. 2A and 2B) of the X-shaped branched DNAs have sticky ends including phosphate groups (P in FIGS. 2A and 2B) at three ends and form a uniform sized DNA nanogel by cross-linking as a result of an enzymatic reaction, for example, by a ligase (FIG. 2A). In another embodiment, m of the X-shaped branched DNAs having phosphate groups at four ends and n of the X-shaped branched DNAs having phosphate groups at three ends cross-link to form a nanogel-sized DNA hydrogel (FIG. 2B). Here, n may be an integer of from about 4 to about 1000, and m may be an integer of from about 4 to about 1000.

After cross-linking the branched DNA, washing with distilled water may be performed. Washing may, for example, remove a salt that may have been added during the cross-linking of the branched DNA, thereby minimizing the content of the salt in the resulting DNA hydrogel. This may maximize the electrostatic attraction between the DNA hydrogel and the nanoparticles, and prevent aggregation of the nanoparticles that may otherwise occur as a result of the presence of the salt.

After cross-linking, or after washing in the event that a step of washing is performed, a step of drying the DNA hydrogel before coupling the nanoparticles may be performed. The drying may be performed by freeze-drying. The freeze-drying may be performed for over twelve hours.

The solution of the nanoparticles having a positively charged surface may be prepared methods known in the art. For example, the solution may be prepared by reacting the nanoparticles with a solution including an amine derivative to couple the cationic ligand to the nanoparticles.

The nanoparticle may be a nanoparticle having a photothermal effect. Also, the nanoparticle may include a metal, a semiconductor, or a quantum dot, as disclosed previously. The metal may be at least one selected from gold, silver, copper, platinum, iron, palladium, and an alloy thereof. The semiconductor may be at least one selected from graphene or a carbon nanotube. The quantum dot may be at least one selected from CdS, CdSe, and CdTe.

The nanoparticles having a positively charged surface may form a cluster by coupling to the DNA hydrogel via, for example, electrostatic attraction. Here, the size of the nanoparticle cluster may vary according to the molar ratio between the nanoparticles and the DNA.

Regarding the DNA hydrogel, the solution including the nanoparticles having a positively charged surface may be added in an amount of from about 0.05 moles to about 10 moles of the nanoparticles with respect to 1 mole of the X-, Y-, or T-DNA building block constituting the DNA hydrogel.

By using a solution including nanoparticles having a positively charged surface in an amount of the above range, a cluster of the nanoparticles having a positively charged surface may be formed that has a size of from about 20 nm to about 500 nm.

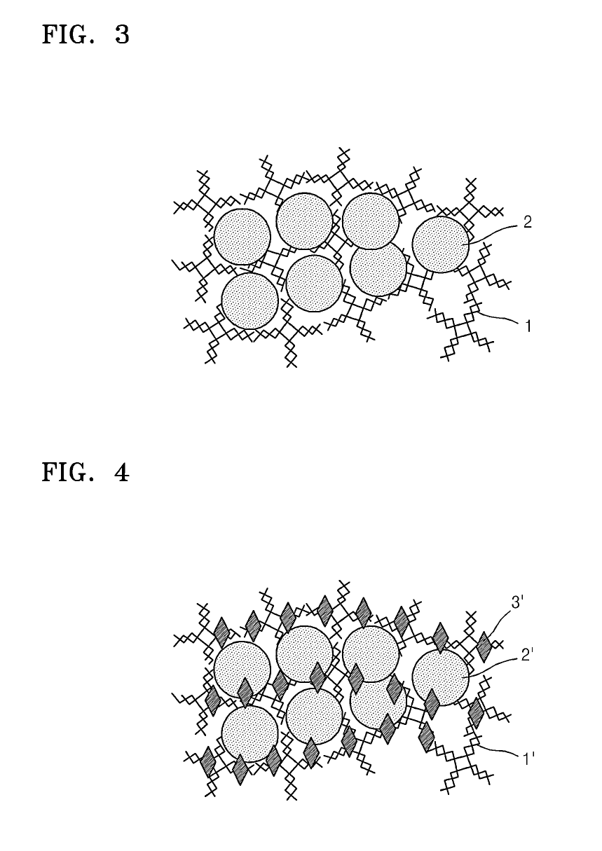

FIG. 3 schematically illustrates a nanoparticle assembly according to an embodiment. As illustrated in FIG. 3, nanoparticles forming clusters may be present in the pores of the DNA hydrogel.

The method according to an embodiment of the present inventive concept, uses, for example, the electrostatic attraction between the surface charge of the nanoparticles and the charge of the DNA hydrogel; hence, the method is applicable to all nanoparticles that are or may be positively charged, regardless of type of the nanoparticles.

The nanoparticle assembly, including the DNA hydrogel and the nanoparticles, prepared according to the method above may be used in various fields, including photothermal treatment, drug delivery, and the like.

According to another aspect, there is provided an active material delivering composite including a DNA hydrogel; nanoparticles having a positively charged surface; and an active material coupled to at least one of the DNA hydrogel or the nanoparticles.

FIG. 4 schematically illustrates an active material delivering composite according to an embodiment.

As illustrated in FIG. 4, nanoparticles forming a cluster 2′ exist in pores of a hydrogel 1′ and an active material 3′, which may be, e.g., a drug, may be coupled to the DNA hydrogel 1′.

According to an embodiment, the active material 3′ may be a drug, contrast media, or recognition/marker material. Examples of the active material include drugs having anti-tumor effects such as doxorubicin, actinomycin D, and the like.

If the active material is a drug, a drug specifically binding to the DNA may be introduced because the active material delivering composite according to an embodiment includes a medium including a DNA. Introducing a drug specifically binding to the DNA may improve treatment efficacy by enabling simultaneous physical and chemical treatments using the nanoparticle assembly. The drug may be any drug without restriction, and may include an intercalating drug, for example, nogalamycin, menogaril; a minor groove binding drug, for example, Hoechst 33258, netropsin, pentamidine, berenil, guanyl bisfuramidine, distamycin, SN7167, SN6999, mithramycin, plicamycin, chromomycin A3; or a covalent cross-linking drug, for example, cisplatin.

Furthermore, an active material delivering composite according to an embodiment of the present inventive concept may naturally be decomposed by deoxyribonuclease or by heat produced via a photothermal effect of the nanoparticles, and the decomposed nanoparticles may be more efficiently excreted out of the body. Accordingly, the active material delivering composite may solve the problem of toxicity resulting an accumulation of nanoparticles in the body, which is the most problematic issue in treatment and diagnosis using nanoparticles.

The DNA hydrogel may be formed by coupling branched DNA. The branched DNA may be an X-shaped branched DNA, a Y-shaped branched DNA, or a T-shaped branched DNA.

The nanoparticles having a positively charged surface may include a metal, a semiconductor, or a quantum dot, as disclosed above. The metal may be at least one selected from gold, silver, copper, platinum, iron, palladium, and an alloy thereof. The nanoparticles may have a diameter of from about 20 nm to about 500 nm.

The nanoparticles may include a cationic ligand.

The nanoparticles having a positively charged surface may be included in an amount of from about 0.05 moles to about 10 moles with respect to 1 mole of an X-DNA in the DNA hydrogel.

The active material delivering composite according to an embodiment has an excellent biocompatibility, exhibits easy coupling and delivery of active materials, and may provide for simultaneous physical and chemical treatment effects.

Hereinafter, specific embodiments are described in greater detail through Examples and Comparative Examples. However, it should be understood that the exemplary embodiments described herein are descriptive only and do not limit the present disclosure.

### Preparation Example 1

### Preparing a DNA Hydrogel

DNA sequences of four ssDNAs for preparing an X-DNA are as follows:

5′-GTGTGTGTGTGTGTGTGTGTGTGTGTGTGTGTGTGTGTGT CG
ACC GAT GAA TAG CGG TCA GAT CCG TAC CTA CTC G-3′
5′-phos-CG AGT AGG TAC GGA TCT GCG TAT TGC GAA
CGA CTC G-3′
5′-phos-CG AGT CGT TCG CAA TAC GGC TGT ACG TAT
GGT CTC G-3′
5′-phos-CG AGA CCA TAC GTA CAG CAC CGC TAT TCA
TCG GTC G-3′

Synthesis of the X-DNA through annealing the four ssDNAs may be performed using a method disclosed in the mentioned above. In other words, each of the four ssDNAs (purchased from IDT) was dissolved in a TE (10 mM Tris-HCl, 1 mM EDTA) buffer solution having a pH of 8.0 to a concentration of 1 mM. 50 uL of each of the resulting solutions was mixed with 300 uL of deionized water to a total amount of 500 uL and annealed to obtain the X-DNA.

By the process above, X-DNA having one deficient sticky end is produced, and the X-DNA was added to 1× ligase buffer solution in the presence of a T4 ligase, maintained at room temperature overnight, salt was removed by using a centrifugal filter to produce a salt-removed reactant, and the salt-removed reactant was resuspended in the same amount of deionized water as used above. Thereafter, the salt-removed reactant was freeze-dried to obtain a DNA hydrogel. The amount of T4 ligase used was 3 units per 30 uL of reaction solution.

Gel electrophoresis was performed by varying concentrations of the X-DNA used during DNA hydrogel synthesis in 40 uM, 120 uM, and 190 uM, respectively. 25/100 bp marker (Bioneer: 25/100 bp DNA ladder) was used, and single stranded DNA (IDT DNA) having 36 bases was used for comparative purposes.

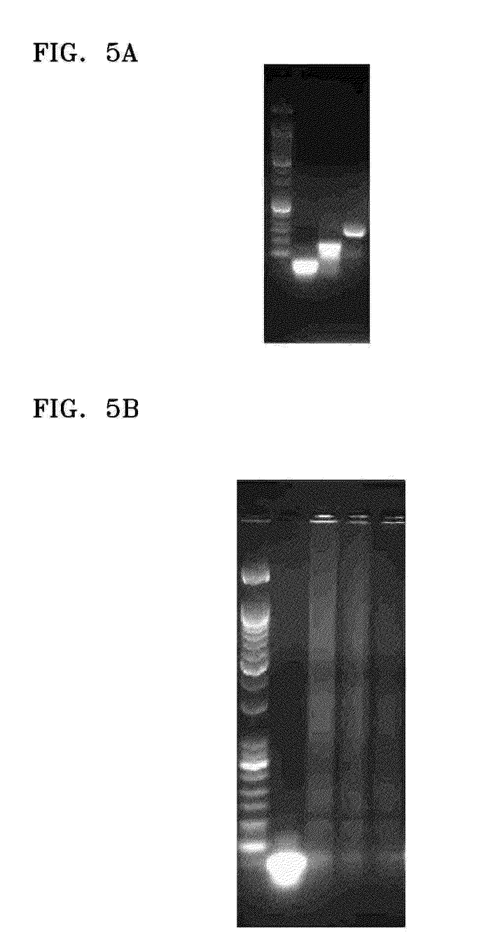

FIGS. 5A and 5B illustrate a result of a gel electrophoresis. As shown in FIGS. 5A and 5B, an X-shaped branched DNA was produced (FIG. 5A), and the X-shaped branched DNA was cross-linked to form a nanogel sized DNA hydrogel (FIG. 5B).

### Preparation Example 2

### Preparing a Negatively Charged (Citrate) Gold Nanoparticle

46.6 mL of deionized water and 3.4 mL of HAuCl₄ solution having a concentration of 14.7 mM were mixed in a round bottom flask to produce a solution. The solution was boiled while being thoroughly agitated to produce a boiling solution. In the boiling solution, 5 mL of sodium citrate having a concentration of 38.8 mM was rapidly added and further boiled for 10 minutes. After heating, the solution was further agitated for 15 minutes and cooled at room temperature.

### Preparation Example 3

### Preparing a Positively Charged Gold Metal Nanoparticle

By adding a small amount of HCl to 100 nM of AuNP citrate (gold nanoparticle), the pH was adjusted to below 2. In 10 mM of a lipoic acid quaternary amine solution, about 3 molar equivalents of dithiothreitol (DTT) was added and reduced for 10 minutes to produce a solution. Excess DTT in the solution was removed by extraction using an ethyl acetate. Equal volumes of the AuNP citrate solution and the lipoic acid quaternary amine solution were mixed and maintained overnight. Thereafter, the concentration of the gold nanoparticle was adjusted to a desired concentration (about 100 nM to about 200 nM) by centrifugation, and a pH of the solution was changed to a deionized state.

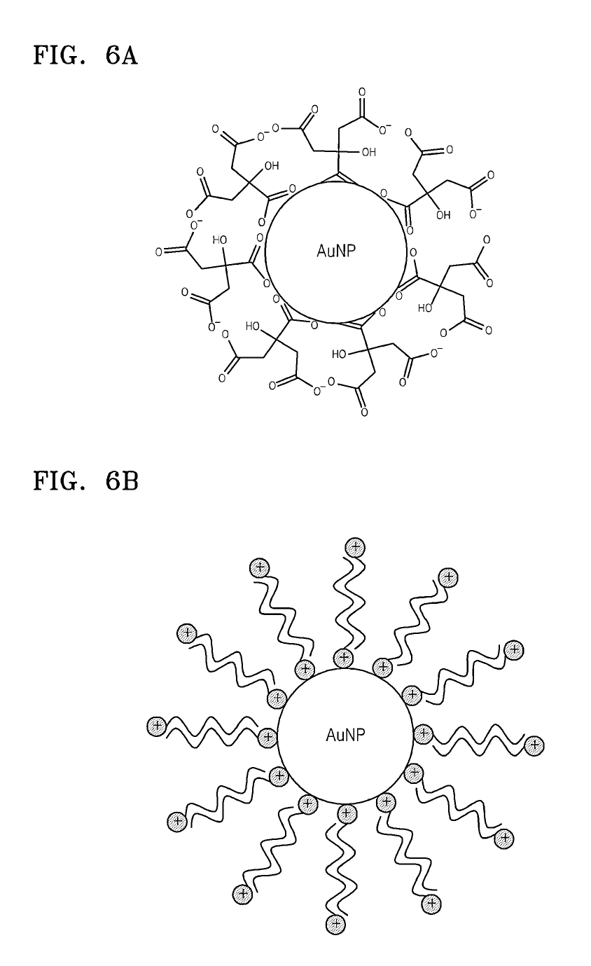

FIG. 6A schematically illustrates a negatively charged gold nanoparticle, and FIG. 6B schematically illustrates a positively charged gold nanoparticle. In FIG. 6A, an overall negative charge is produced as negative ion parts of the ligands are located on an outer surface of the gold nanoparticle.

In FIG. 6B, an overall positive charge is produced as positively charged parts of surfactants are all located on a surface and the outer surface of the gold nanoparticle.

To test whether the nanoparticle may bind to the DNA hydrogel prepared in Preparation Example 1 by electrostatic attraction, a test was performed as follows:

In each of the three microtubes for centrifugation, 60 uL of a dried DNA hydrogel prepared in Preparation Example 1 (in this case, the concentration of the X-DNA was 150 uM) was added. A positively charged and negatively charged quantum dot (CdSe/CdZnS core-shell quantum dot), a negatively charged quantum dot, and a negatively charged quantum dot were each added in a concentration of 200 nM and in an amount of 100 uL to the microtubes and left for 12 hours.

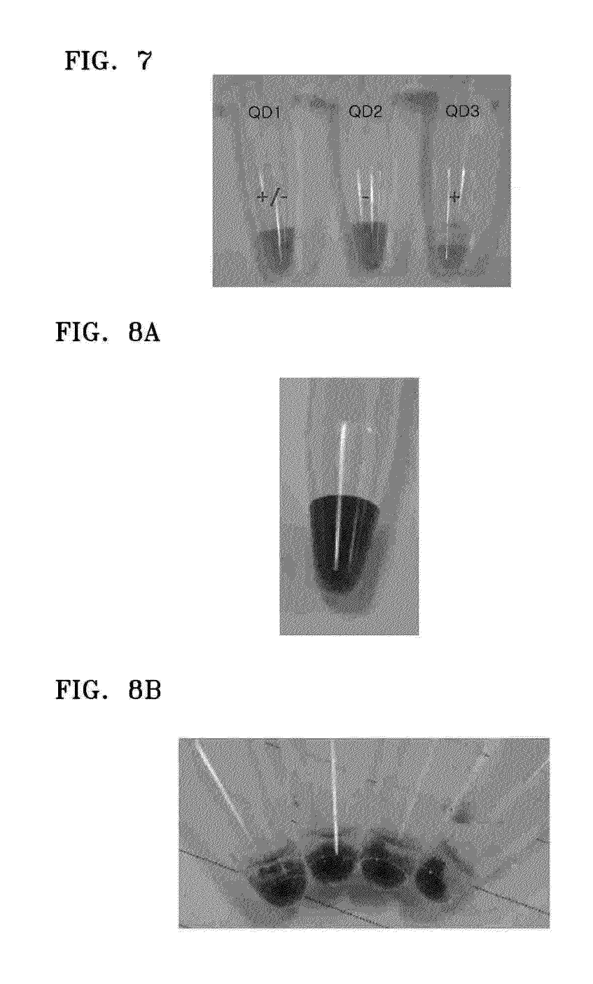

FIG. 7 illustrates a result of coupling nanoparticles to the DNA hydrogel according to surface charges of the quantum dots created after 12 hours. As illustrated in FIG. 7, when the positively charged quantum dots (QD3) were added to the DNA hydrogel, a separation of layers was observed, unlike when positively charged and negatively charged quantum dots were added together (QD1) and when negatively charged quantum dots were added (QD2). The separation of layers happens because the positively charged quantum dots couple to the DNA hydrogel due to electrostatic attraction, creating a nanoparticle assembly, and sinking to the bottom due to the heavy weight of the nanoparticle assembly.

Accordingly, the nanoparticle assembly may be formed by coupling the nanoparticles having a positively charged surface to the DNA hydrogel.

### Preparing a Nanoparticle Assembly of Gold Nanoparticles+DNA Hydrogel

### Example 1

By using the DNA hydrogel (X-DNA 190 uM) obtained in Preparation Example 1 and the gold nanoparticles having a positively charged surface obtained in Preparation Example 3 were used to prepare a nanoparticle assembly as follows:

In the DNA hydrogel obtained in Preparation Example 1, a solution (200 nM and 100 uL), including the gold nanoparticles having a positively charged surface obtained in Preparation Example 3, was added. After leaving the resulting solution at room temperature for 24 hours, supernatant liquid was removed by centrifuging the solution for 10 minutes at 5000 rpm, and a nanoparticle assembly in which the gold nanoparticles are coupled to the DNA hydrogel was obtained by resuspending a precipitate using deionized water.

### Example 2

The same method as in Example 1 was used to prepare the nanoparticle assembly except for varying the concentration of the X-DNA in the DNA hydrogel to 150 uM.

### Example 3

The same method as in Example 1 was used to prepare the nanoparticle assembly except for varying the concentration of the X-DNA in the DNA hydrogel to 112 uM.

### Example 4

The same method as in Example 3 was used to prepare the nanoparticle assembly except for varying the concentration of the gold nanoparticles to 100 nM.

### Example 5

The same method as in Example 3 was used to prepare the nanoparticle assembly except for varying the concentration of the gold nanoparticles to 50 nM.

### Comparative Example 1

The same method as in Example 1 was used to prepare the nanoparticle assembly except for using the negatively charged gold nanoparticles in Preparation Example 2 instead of the positively charged gold nanoparticles in Preparation Example 3.

The nanoparticle assemblies prepared in Examples 1 to 3 and Comparative Example 1 were added to tubes, and observed.

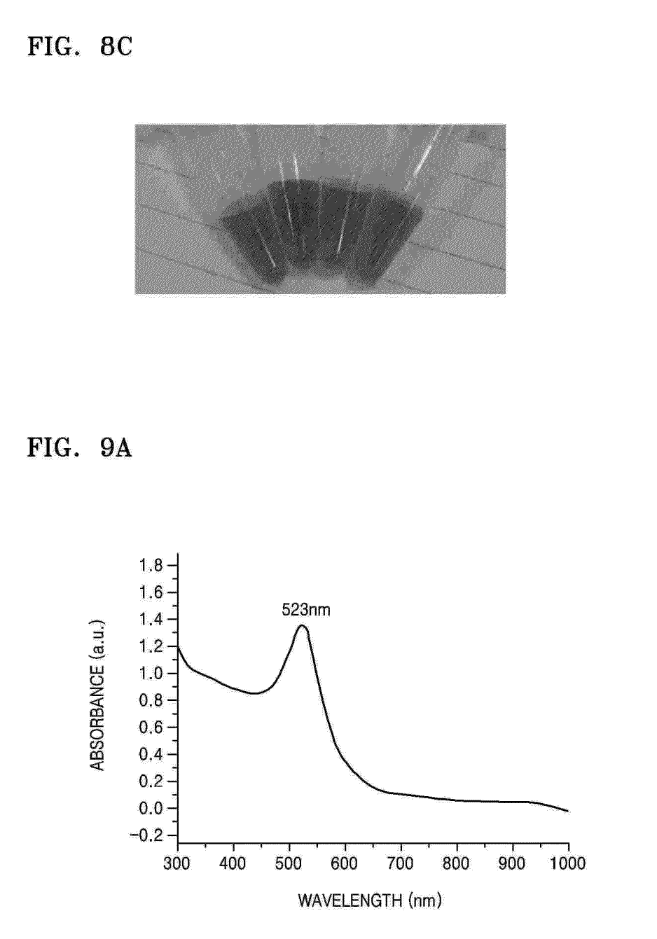

FIGS. 8A and 8B each illustrate the results of the nanoparticle assembly. In FIG. 8A (Comparative Example 1), the entire solution exhibits a uniform color because a coupling between the DNA hydrogel and the negatively charged gold nanoparticles did not occur. On the other hand, in FIG. 8B (Examples 1 to 3), the separation of layers is shown because of a coupling between the DNA hydrogel and the positively charged gold nanoparticles. FIG. 8C illustrates products of agitating and dissolving the products of FIG. 8B in an aqueous solution. As shown in FIG. 8C, the extent of the aggregation of particles in the solution varies according to the concentration ratio of the concentration of the positively charged gold nanoparticles and the concentration of the X-DNA in the DNA hydrogel.

An absorption spectrum with respect to the nanoparticle assembly obtained in Examples 1 to 3, and Comparative Example 1 was observed.

FIG. 9A is an absorption spectrum of the gold nanoparticles only, and FIG. 9B is an absorption spectrum of the nanoparticle assembly prepared in Examples 1 to 6, and Comparative Example 1.

As illustrated in FIGS. 9A and 9B, the absorption spectrum of the nanoparticle assembly, including the negatively charged gold nanoparticles (Comparative Example 1), is the same as the absorption spectrum of the gold nanoparticles only; hence, the size of the gold nanoparticles did not change. On the other hand, the nanoparticle assembly including the positively charged gold nanoparticles (Examples 1 to 3) exhibit a change in the absorption spectrum because the gold nanoparticles form a cluster due to electrostatic attraction of the DNA hydrogel. Hence, the extent of absorption with respect to a long wavelength increased. Also, as a comparative amount of the gold nanoparticles increased, the extent of absorption with respect to the long wavelength increased.

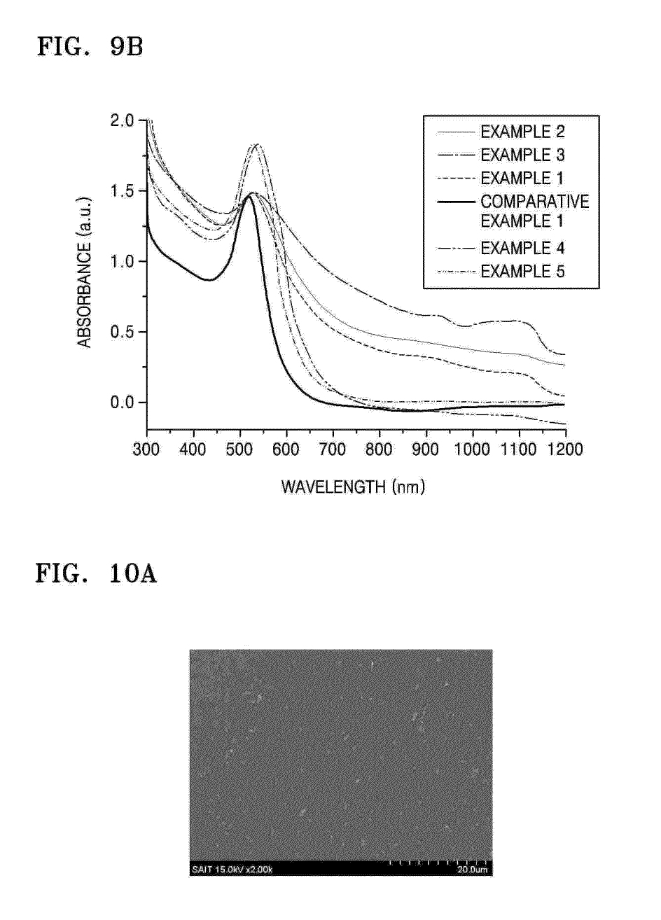

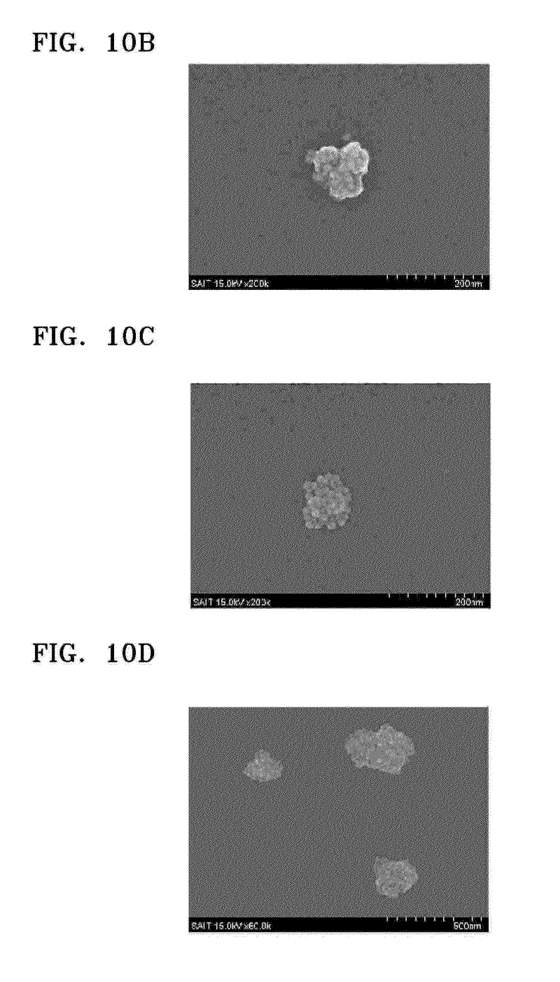

FIGS. 10A through 1D are scanning electron microscope images of the nanoparticle assembly prepared according to Example 2 of the present inventive concept. Images have magnifications of 2000× (FIG. 10A), 200,000× (FIG. 10B), 200,000× (FIG. 10C), and 800,000× (FIG. 10D), respectively. As shown in FIG. 10, the nanoparticle assembly prepared according to the method of the present inventive concept exists in a cluster form.

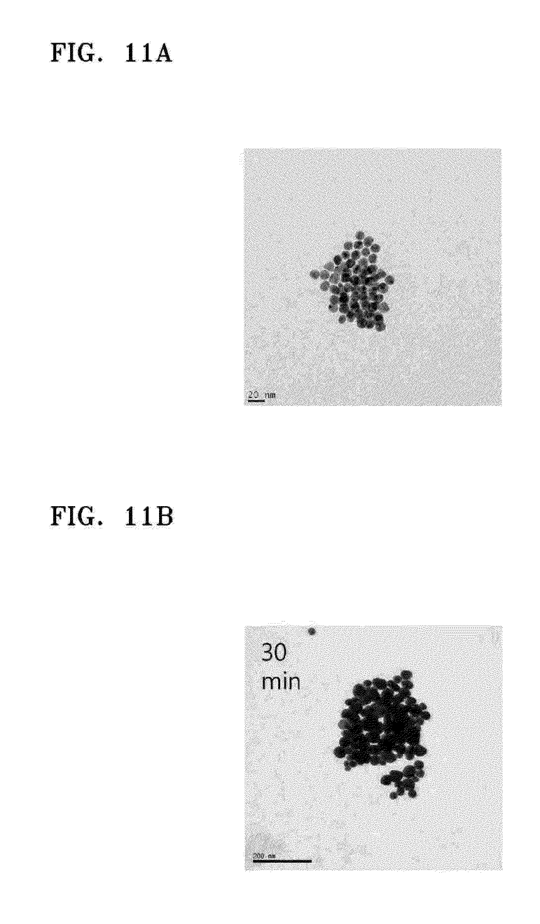

FIGS. 11A and 11B are transmission electron microscope (TEM) images of the nanoparticle assembly prepared according to Example 2. For comparative purposes, a TEM image of aggregated gold nanoparticles after adding an excess amount of salt (100 mM NaCl) to the solution including the positively charged gold nanoparticles is shown. As illustrated in FIG. 11A, the nanoparticle assembly prepared according to a method above forms a cluster, maintaining gaps between each nanoparticles, whereas the gold nanoparticles prepared according to a method of aggregating the gold nanoparticles by using a salt do not have gaps between each nanoparticle and exist as an aggregate (see FIG. 11B). Hence, the nanoparticle assembly according to an embodiment illustrates an optical activity similar to nanoparticles having a size greater than 100 nm and may be easily decomposed into each nanoparticle for easy excretion out of the body after activity; however, when the gold nanoparticles exist as an aggregate, decomposition of the gold nanoparticles is difficult; hence, the gold nanoparticles may accumulate in the body and create toxicity.

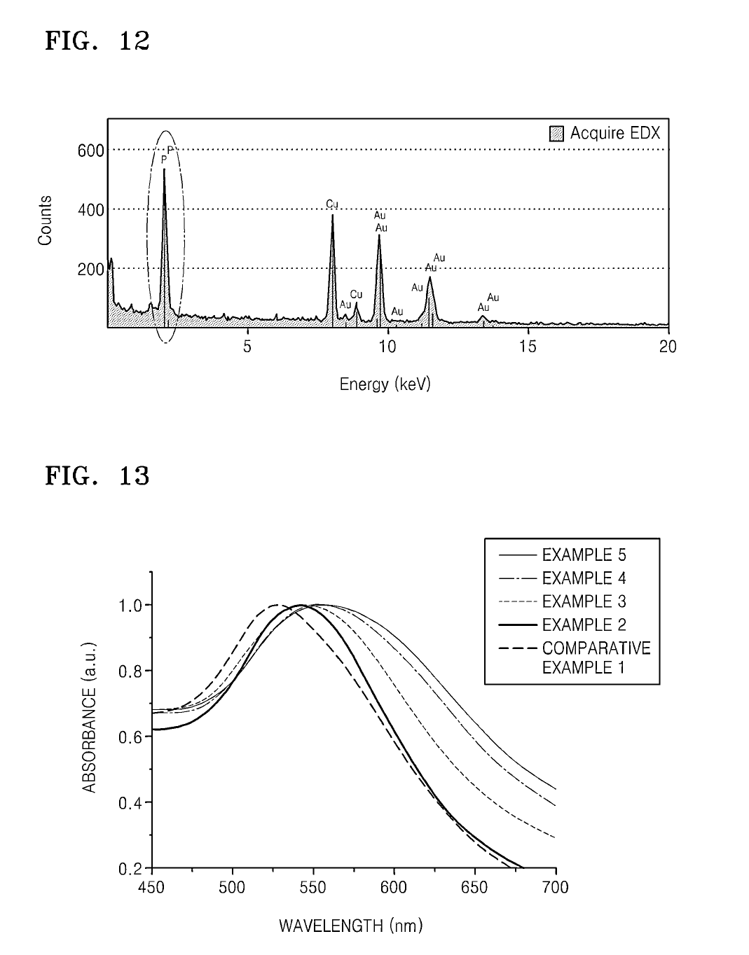

The nanoparticle assembly obtained according to Example 2 was analyzed by energy dispersive spectroscopy, and a result thereof is illustrated in FIG. 12. As illustrated in FIG. 12, a gold element and a phosphor element were detected together, showing that the DNA hydrogel and the gold nanoparticles are coupled.

FIG. 13 illustrates a detailed absorption spectrum of the nanoparticle assembly prepared in Examples 2 and 3 in a narrower range of wavelengths. In FIG. 13, GT40 corresponds to Example 2, and GT30 corresponds to Example 3. GT10 corresponds to Example 5, and GT20 corresponds to Example 4. Also in FIG. 13, the extent of clustering of the nanoparticles in the solution varies according to the concentration ratio between the concentration of the positively charged gold nanoparticles and the concentration of the X-DNA in the DNA hydrogel.

The size of the cluster of the nanoparticles in the nanoparticle assembly prepared in Examples 1 to 3 was measured by using dynamic light scattering, and a result thereof is shown in Table 1.

TABLE 1

| Example number | Average diameter (nm) |
|---|---|
| Example 1 | 169 |
| Example 2 | 216 |
| Example 3 | 255 |
| Example 4 | 269 |

As illustrated in the Table 1 above, the nanoparticles in the nanoparticle assembly according to an embodiment exist in a cluster form.

Photothermal Test of Nanoparticle Assembly

The prepared assembly was made into an aqueous solution of a prescribed concentration (based on nanoparticles), the aqueous solution was added to a cell culture plate where cells are bonded, and delivery of the particles into the cells was measured according to time. After sufficient time to deliver particles into the cells, a laser light having a wavelength of 660 nm was irradiated at a certain strength for a certain amount of time. After irradiating laser light, the cells were dyed to confirm apoptosis.

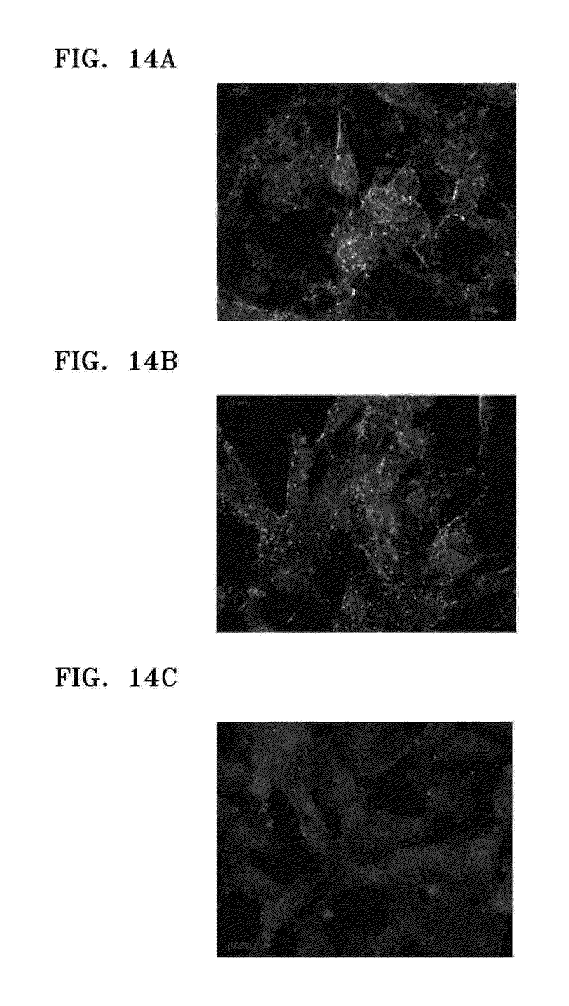

FIGS. 14A through 14C are dark field microscopic images illustrating a delivery of an assembly of the gold nanoparticles in the DNA hydrogel into the cells. FIG. 14A corresponds to a concentration of the gold nanoparticles of 5 nM, FIG. 14B corresponds to a concentration of 3 nM, and FIG. 14C corresponds to a concentration of 1 nM.

As illustrated in FIGS. 14A through 14C, a sufficient delivery into the cells may be achieved when the concentration of the gold nanoparticles is greater than or equal to 5 nM, and the photothermal effect increases as the concentration of the gold nanoparticles increases.

### Drug Delivery Effect Test

### Example 6

As the drug, the antitumor agent doxorubicin was used.

The antitumor agent doxorubicin in an aqueous solution state was added to a solution of the gold particles assembled in the DNA hydrogel where the gold nanoparticles were present in an amount of about 500 times more than the doxorubicin to produce a solution. Thereafter, the solution was centrifuged to remove residual doxorubicin. Photothermal treatment and the chemical treatment effects were confirmed by delivering the gold nanoparticle/DNA hydrogel assembly including the doxorubicin into the cells by using the same method as in Example 5.

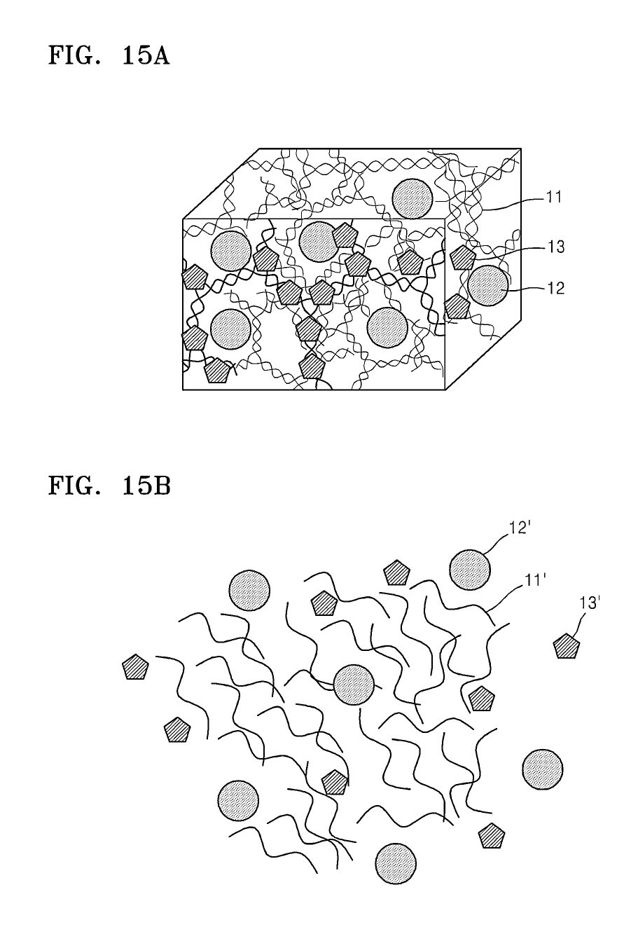

FIGS. 15A and 15B schematically illustrate coupling and excretion of active materials according to an embodiment, in particular, a drug-containing embodiment. The nanoparticles 12, for example, gold nanoparticles, couple to the DNA hydrogel 11 to form a cluster, and the active material 13 doxorubicin couples to the DNA hydrogel 11 to form a drug delivering composite (FIG. 15A). When light is irradiated on the drug delivering composite, the temperature is increased as heat is produced due to the photothermal effect. As a result, a cross-link between the DNA hydrogel 11′ decomposes, thus destroying the cluster of the gold nanoparticles 12′, creating individual nanoparticles, decoupling the doxorubicin 13′ from the DNA, and excreting the doxorubicin to a desired region.

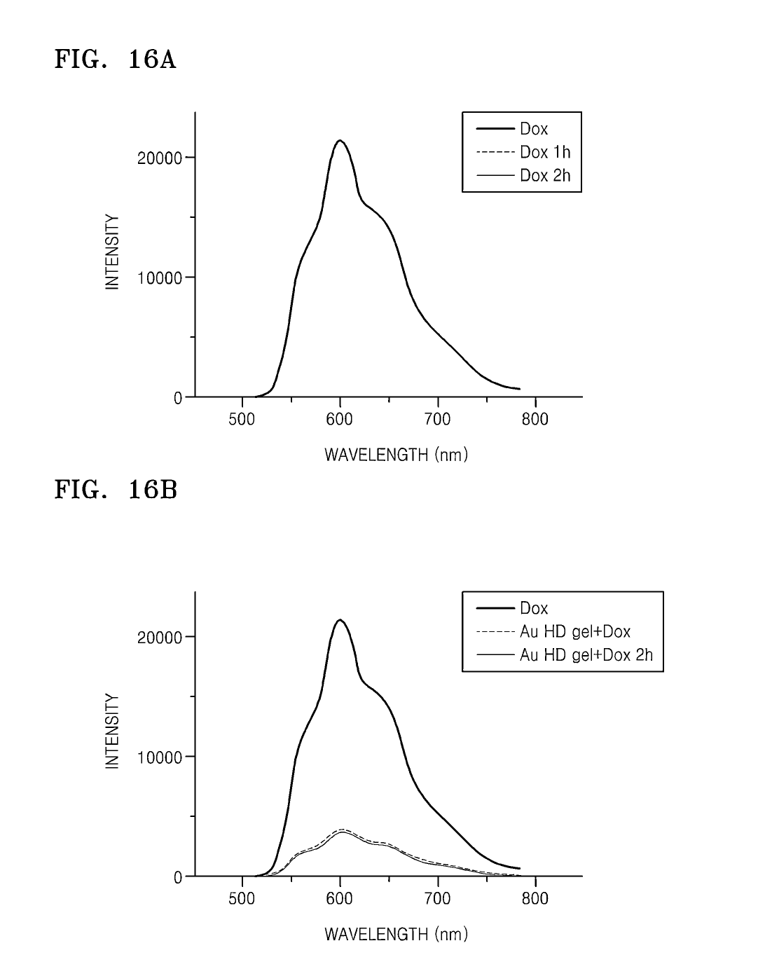

FIGS. 16A and 16B are graphs illustrating a measurement of a decrease in a fluorescence of the drug delivering composite prepared in Example 6.

FIG. 16A is a graph illustrating the measurement of a change in fluorescence of a doxorubicin solution according to time, and FIG. 16B is a graph illustrating the measurement of changes in fluorescence of the doxorubicin solution and the drug delivering composite according to an embodiment. As illustrated in FIGS. 16A and 16B, the fluorescence of the drug delivering composite, according to an embodiment, was substantially reduced as the doxorubicin and the nanoparticle assembly became closer to each other, as a result of coupling and quenching occurring. However, a reduction in fluorescence of doxorubicin alone was not observed when measured as a function of time.

Size and Zeta Potential Measurement of Hydrated Particle

The size and zeta potential of the hydrated particles of the drug delivering composite prepared in Example 6 and the nanoparticle assembly before coupling to the drug were measured and results are shown in Table 2.

TABLE 2

| | Hydrated particle size | Zeta potential |
|---|---|---|
| | (nm) | (mV) |
| Nanoparticle assembly | 45.7 (±10.6) | −41.2 (±4.33) |
| (Example 6) | | |
| Drug delivering composite | 69.9 (±10.7) | −40.0 (±0.29) |
| (Example 6) | | |

As shown in Table 2, the drug is well disposed between the nanoparticle assemblies.

The exemplary embodiments provide a nanoparticle assembly having excellent biocompatibility that is capable of easily excreting nanoparticles out of a body easily coupling and delivering active materials.

It should be understood that the exemplary embodiments described herein should be considered to be descriptive only and do not limit the scope of the present disclosure. Descriptions of features or aspects within each embodiment should typically be considered as being available for other similar features or aspects in other embodiments.

What is claimed is:

1. A nanoparticle assembly comprising:
a DNA hydrogel; and
nanoparticles having a positively charged surface, wherein the nanoparticles are coupled to the DNA hydrogel by electrostatic attraction.

2. The nanoparticle assembly of claim 1, wherein the DNA hydrogel is formed by cross-linking at least one selected from the group consisting of X-DNA, Y-DNA, and T-DNA.

3. The nanoparticle assembly of claim 1, wherein the DNA hydrogel is a nanogel having a diameter of from about 50 nm to about 500 nm.

4. The nanoparticle assembly of claim 1, wherein the nanoparticles having the positively charged surface have a size of from about 10 nm to about 50 nm.

5. The nanoparticle assembly of claim 1, wherein a size of the nanoparticle assembly is from about 50 nm to about 500 nm.

6. The nanoparticle assembly of claim 1, wherein the nanoparticles having the positively charged surface comprise a metal, a semiconductor, or a quantum dot.

7. The nanoparticle assembly of claim 6, wherein the metal comprises at least one of gold, silver, copper, platinum, iron, palladium, or an alloy thereof.

8. The nanoparticle assembly of claim 1, wherein the nanoparticles having the positively charged surface have a photothermal effect.

9. The nanoparticle assembly of claim 1, wherein the nanoparticles having the positively charged surface comprise a cationic ligand.

10. The nanoparticle assembly of claim 9, wherein the cationic ligand is at least one of a macromolecule, a cationic surfactant, and an amine derivative.

11. The nanoparticle assembly of claim 2, wherein the nanoparticles having the positively charged surface are present in an amount of from about 0.05 moles to about 10 moles with respect to 1 mole of X-DNA in the DNA hydrogel.

12. A method of preparing a nanoparticle assembly, the method comprising:
preparing a DNA hydrogel;
preparing a solution comprising nanoparticles having a positively charged surface; and
coupling the nanoparticles having the positively charged surface and the DNA hydrogel by adding the solution to the DNA hydrogel.

13. The method of preparing the nanoparticle assembly of claim 12, wherein the preparing the DNA hydrogel comprises:
hybridizing single-stranded DNAs to prepare a branched DNA; and
cross-linking the branched DNA.

14. The method of preparing the nanoparticle assembly of claim 13, wherein the branched DNA comprises a DNA having a partly deficient sticky end.

15. The method of preparing the nanoparticle assembly of claim 13, wherein the cross-linking of the branched DNA is performed until the DNA hydrogel becomes a nanogel having a size of from about 50 nm to about 500 nm.

16. The method of preparing the nanoparticle assembly of claim 13, wherein the branched DNA is cross-linked by a DNA ligase.

17. The method of preparing the nanoparticle assembly of claim 12, wherein the preparing the solution comprising the nanoparticles having a positively charged surface comprises: preparing nanoparticles; and coupling a cationic ligand to the nanoparticles by reacting the nanoparticles with a solution comprising an amine derivative.

18. The method of preparing the nanoparticle assembly of claim 12, wherein X-DNA is present in the DNA hydrogel, and the solution comprising the nanoparticles having the positively charged surface is added to the DNA hydrogel in an amount of from about 0.05 moles to about 10 moles of the nanoparticles having the positively charged surface with respect to 1 mole of X-DNA in the DNA hydrogel.

19. The method of preparing the nanoparticle assembly of claim 18, wherein the nanoparticles having the positively charged surface are assembled as a cluster having a size of from about 50 nm to about 500 nm in the DNA hydrogel.

20. A drug delivering composite, the composite comprising:
a DNA hydrogel;
nanoparticles having a positively charged surface; and
a drug coupled to at least one of a DNA hydrogel or nanoparticles;
wherein the nanoparticles are coupled to the DNA hydrogel by electrostatic attraction.

21. A method of treating cancer comprising administering the nanoparticle assembly of claim 1 to a subject in need thereof.

22. A method of treating cancer comprising administering the nanoparticle assembly of claim 8 to a subject in need thereof; and eliciting said photothermal effect.

23. A method of removing the nanoparticle assembly of claim 8 from a body comprising eliciting said photothermal effect.
